(19) United States
(12) Patent Application Publication
(10) Pub. No.: US 2017/0029802 A1
(43) Pub. Date: Feb. 2, 2017

(54) ACOUSTIC AFFINITY SEPARATION

(71) Applicant: FloDesign Sonics, Inc., Wilbraham, MA (US)

(72) Inventors: Bart Lipkens, Hampden, MA (US); Rudolf Gilmanshin, Framingham, MA (US); Walter M. Presz, Wilbraham, MA (US); Louis Masi, Wilbraham, MA (US); Thomas J. Kennedy, III, Wilbraham, MA (US)

(21) Appl. No.: 15/222,800

(22) Filed: Jul. 28, 2016

Related U.S. Application Data
(60) Provisional application No. 62/197,801, filed on Jul. 28, 2015.

(51) Int. Cl. C12N 13/00; G01N 30/72; G01N 30/46; G01N 30/96; C12M 1/42; C12M 1/00

(52) U.S. Cl. CPC C12N 13/00; C12M 35/04; C12M 47/02; G01N 30/462; G01N 30/96; G01N 30/72; G01N 2030/027

(57) ABSTRACT

Methods and systems for separating a first biomaterial from a second biomaterial can use functionalized material retained in a liquid-filled chamber at locales within an acoustic standing wave field. A culture suspension containing the first biomaterial and the second biomaterial flows into the liquid-filled chamber and at least portions of the first biomaterial with features complementary to the functionalized material becomes bound to the functionalized material while other portions of the culture suspension containing the second material pass through the chamber. The portion of the first biomaterial bound to the functionalized material is subsequently released from the liquid filled chamber.

### TECHNICAL FIELD

This disclosure relates to separation of biomaterials.

### BACKGROUND

Separation of biomaterial has been applied in a variety of contexts. For example, separation techniques for separating proteins from other biomaterials are used in a number of analytical processes.

### SUMMARY

This disclosure describes technologies relating to methods, systems, and apparatus for separation of biomaterials accomplished by functionalized material distributed in a fluid chamber that bind the specific target materials such as recombinant proteins and monoclonal antibodies. The functionalized material, such as microcarriers that are coated with an affinity protein, is trapped by nodes and anti-nodes of an acoustic standing wave. In this approach, the functionalized material is trapped without contact (for example, using mechanical channels, conduits, tweezers, etc.).

In one aspect, some methods of performing chromatography analysis of samples include: retaining functionalized material in a liquid-filled chamber at locales within an acoustic standing wave field, the locales distributed inside the chamber where acoustic pressure amplitude is either elevated compared to when the acoustic transducer is turned off, or substantially identical to when the acoustic transducer is turned off; flowing fluid containing the samples into the liquid-filled chamber where functionalized material has been retained by acoustic insonification such that a portion of the samples with features complementary to the functionalized material become bound to the functionalized material while other portions of the samples pass through the chamber; and subsequently processing fluid inside the chamber to cause the portion of samples that are bound to the functionalized material retained therein to elute from the chamber. Implementations may include one or more of the following features.

The method may include causing the portion of samples to elute from the chamber and into an analysis bin.

Processing fluid inside the chamber may include: passing the fluid through a size exclusion column wherein protein samples of a first hydrodynamic radius elutes before samples with a second hydrodynamic radius when the first hydrodynamic radius is larger than the second hydrodynamic radius.

Processing fluid inside the chamber may include: increasing an ionic strength of the fluid to cause the portion of samples that are bound to the functionalized material to elute.

Processing fluid inside the chamber may include: adjusting a pH level of the fluid to cause the portion of samples that are bound to the functionalized material to elute.

Processing fluid inside the chamber further may include: lowering an ionic strength of the fluid to cause the portion of samples that are bound to the functionalized material to refold into a native formation such that a hydrophobic interaction between the portion of samples and the functionalized material is decreased.

The method may include determining a quantitative level of the portion of samples eluted to the analysis bin to form a chromatography readout. The method may include determining the quantitative level comprises determining a mass or a volume. Determining the quantitative level may include measuring an optical absorption index of the portion of samples in the analysis bin.

In some embodiments, the portion of the samples form antigen-antibody interactions with binding sites on the functionalized material. The portion of the samples become bound to the functionalized material when a ligand of the portions of the samples is conjugated to a matrix on the functional material. The functionalized material include functionalized microbeads. The functionalized microbeads include a particular antigen ligand that has affinity for a corresponding antibody.

In some embodiments, flowing the fluid containing the protein samples into the liquid-filled chamber includes: circulating the fluid containing the protein samples such that the samples are flown more than once through the locales distributed inside the chamber where acoustic pressure amplitude is either elevated compared to when the acoustic transducer is turned off, or substantially identical to when the acoustic transducer is turned off.

In some embodiments, the samples are protein samples. The samples include target compounds, such as recombinant proteins and monoclonal antibodies, viruses, and live cells (e.g., T cells).

Some apparatus for chromatography analysis include: a flow chamber having a first wall and a second wall opposite to each other, and configured to receive fluid containing functionalized material; an acoustic transducer mounted on the first wall and a reflector mounted on the second wall such that when the acoustic transducer is turned on, a multi-dimensional acoustic field is created inside the chamber that includes first spatial locales where acoustic pressure amplitude is elevated from when the acoustic transducer is turned off, and second spatial locales where acoustic pressure amplitude is substantially identical to when the acoustic transducer is turned off wherein functional material become trapped at the first or second locales of the multidimensional acoustic field; and an inlet coupled to the flow chamber and configured to flow protein samples through the flow chamber where functionalized material is trapped such that a portion of the protein samples with features complementary to the functionalized material become bound to the functionalized material while other portions of the protein samples and other materials such as cell debris pass through the flow chamber. Implementations may include one or more of the following features.

The apparatus may include an analysis bin configured to receive the portion of the protein samples bound to the functionalized material and subsequently eluted from the functionalized material such that a chromatography measurement of the portion of the protein samples is obtained.

The apparatus may further include: a size exclusion column coupled to the flow chamber and configured to cause the portion of the protein samples bound to the functionalized material to elute from the functionalized material.

The apparatus may further include a hydrophobic interaction chromatography column coupled to the flow chamber and configured to cause the portion of the protein samples bound to the functionalized material to elute from the functionalized material.

The apparatus may further include: an ion exchange chromatography column coupled to the flow chamber and configured to cause the portion of the protein samples bound to the functionalized material to elute from the functionalized material.

The apparatus may further include: a mass spectrometer to measure an amount of the portion of the protein samples in the analysis bin.

The apparatus may further include an optical spectrometer to measure an amount of the portion of the protein samples in the analysis bin.

The functionalized microcarriers may also be circulated after the recombinant proteins or monoclonal antibody is eluted from the surface by a buffer or other process elution. This allows for greater surface area and affinity interaction of the functionalized microcarriers with the expressed proteins from the bioreactor, increasing the efficiency of the acoustic fluidized bed chromatography process.

The apparatus provides functionalized particles in an arrangement that provides more space between particles than packed columns. The lower density decreases the likelihood that non-target biomaterials will clog flow paths between the functionalized particles. Recirculating media containing the target biomaterials in effect increases the capture surface area of the apparatus by passing free target biomaterials past the functionalized particles multiple times. The reduced contact of non-target biomaterials such as cells can help preserve the viability of cells being used to produce, for example, proteins. The technology described here can be used in high density cell culture, new research applications, large production culture volumes, e.g., more than 1,000 liters, efficient monitoring and culture control, reduction of costs and contamination in cell culture applications.

The details of one or more implementations of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1A is schematic view of a system using functionalized material held in an acoustic affinity filter to capture biomaterials produced in a bioreactor.

FIG. 1B is a schematic diagram showing a portion of an affinity chromatography system of FIG. 1A using a bed of functionalized material distributed in a fluid chamber such that the bed of functionalized material to binds specific proteins.

FIGS. 1C-1E show the system of FIG. 1A during operation.

FIG. 2 is a flow chart of a process for extracting protein samples from a fluid as input to chromatography and into analysis bins.

FIG. 3 is a photograph showing an example bed of microbeads distributed in a fluid chamber and trapped at the nodes and anti-nodes of a multi-dimensional acoustic wave created in the fluid chamber.

FIG. 4 is a flowchart of a process in which functionalized material is incubated directly in a cell culture suspension within a bioreactor.

FIG. 5 is a flowchart of a process in which the slurry is loaded in a chromatography column and processed in the way similar to a regular chromatographic procedure

FIG. 6 is a flowchart of a process in which an acoustic affinity filter with the microbeads inside can be used similarly to a chromatography column in a dedicated cycle.

FIG. 7 illustrates using size exclusion chromatography to extract and analyze proteins from a fluid.

FIG. 8 illustrates using ion exchange chromatography to extract and analyze proteins from a fluid.

FIG. 9 is a schematic of a system for producing monoclonal antibodies and recombinant proteins.

FIGS. 10A and 10B are schematics of a system for producing monoclonal antibodies and recombinant proteins.

Like reference numbers and designations in the various drawings indicate like elements.

### DETAILED DESCRIPTION

This disclosure describes methods, systems and apparatus for retaining functionalized materials in an acoustic standing wave distribution with nodes and antinodes that trap the functionalized materials. The functionalized materials includes binding agents with particular affinity to selected biomaterials such as, for example, biomolecules (i.e., proteins, lipids, carbohydrates, and nucleic acids), viruses, virus-like particles, vesicles, and exosomes.

(e.g., selected proteins, biomolecules, macromolecules, and supramolecular structures). The acoustic standing wave field distribution can retain the functionalized materials (e.g., chromatographic beads) without contact or physical support at locations inside a fluid chamber.

The non-invasive manner in which the functionalized material is retained in the fluid chamber creates an in-situ matrix structure. By flowing cellular samples through this matrix structure, biomaterials with features complementary to the retained functionalized material can be bound to the functionalized material while other materials pass through the fluid chamber. Subsequently, the fluid containing the functionalized material with attached biomaterials can be further processed to extract the biomaterials.

In some systems, proteins with complementary features can bind to the functionalized material while other proteins and/or cellular components pass through. This process allows for selective trapping and separation of specific ligands, proteins, antibodies, free DNA, viruses, or cells, or of any object conjugated with a complementary determinants, while other particulates that are in the fluid stream are allowed to flow past the acoustic standing wave with the trapped functionalized material (e.g., particles and beads).

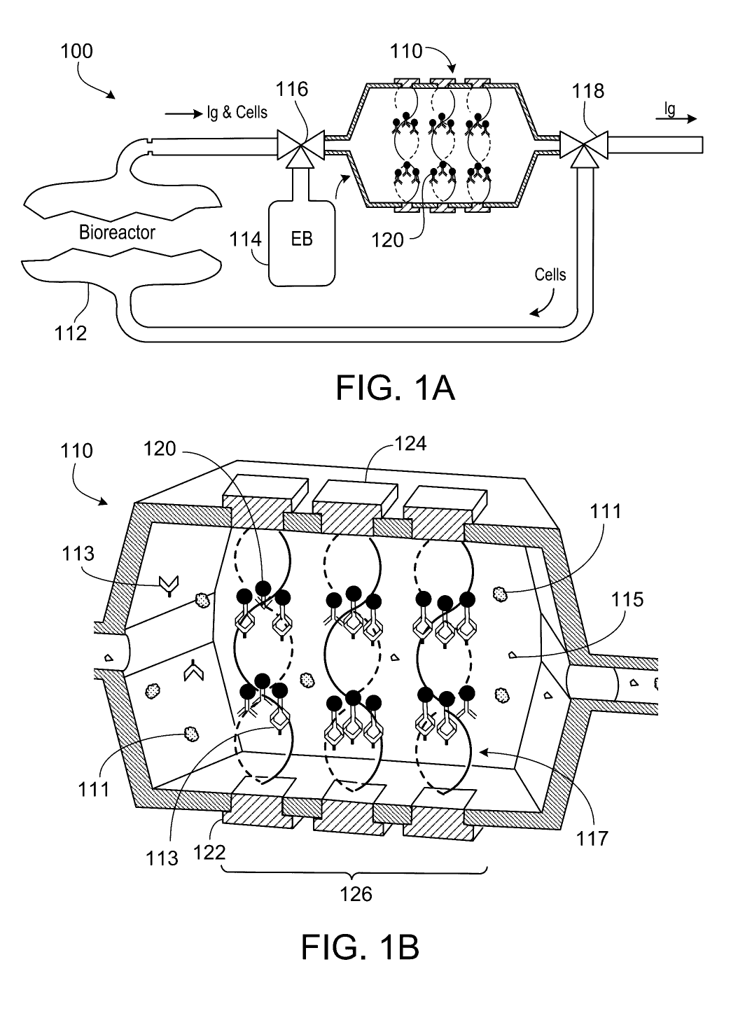

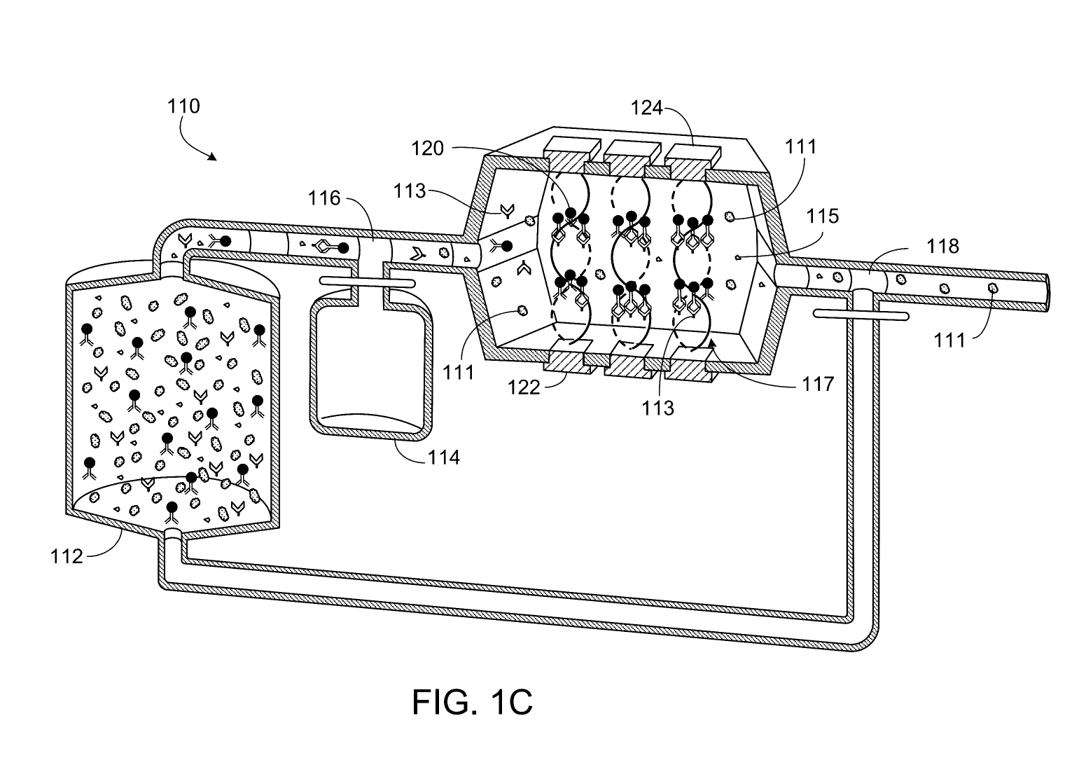

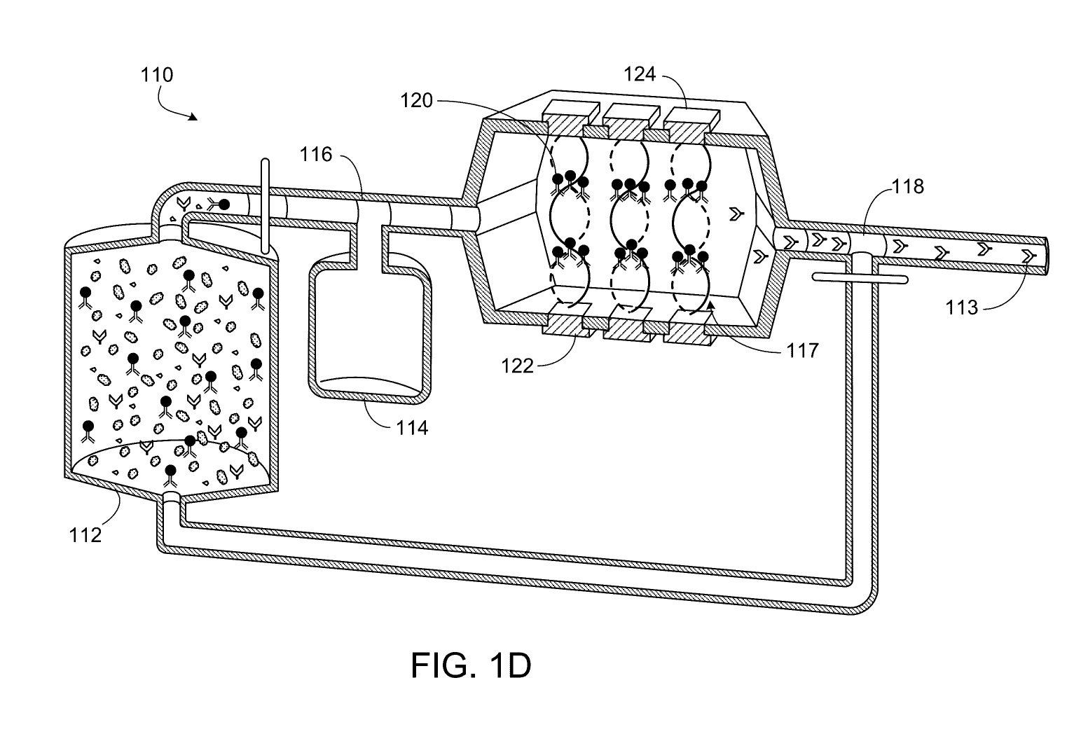

FIG. 1A illustrates a system 100 that uses functionalized material as part of an acoustic affinity filter 110 to capture materials produced in a bioreactor 112. The system 100 includes the acoustic affinity filter 110, the bioreactor 112, and an elution buffer reservoir 114. The bioreactor 112 is operated to cause cells 111, which may be Chinese Hamster Ovary (CHO) cells, for example, (see FIGS. 1B-1E) contained in the bioreactor 112 to produce materials 113, which may be monoclonal antibodies or recombinant proteins, for example (see FIGS. 1B-1E). The system 100 extracts the materials 113 by passing fluid containing the cells 111 and the materials 113 through the acoustic affinity filter 110. The acoustic affinity filter 110 retains the materials 113 while the cells 111 and debris/non-target components 115 (see FIGS. 1B-1E) pass through. A three-way valve 116 provides a controllable connection between an outlet of the bioreactor 112, an outlet of the elution buffer reservoir 114, and an inlet of the acoustic affinity filter 110. Another three-way valve 118 provides a controllable connection between an inlet of the bioreactor 112, an outlet of the acoustic affinity filter 110, and an outlet of the system 100. The acoustic affinity filter 110 can be preloaded with microbeads 120 of chromatography resin that has affinity to the materials being produced (see FIG. 1B) or the microbeads 120 can be present in the bioreactor 112 during incubation (see FIGS. 1C-1E).

FIG. 1B is a schematic diagram illustrating structure and functionality of the acoustic affinity filter 110 in more detail. The acoustic affinity filter 110 includes an acoustic transducer 122 and a reflector 124. The acoustic transducer 122 and the reflector 124 are mounted on opposite walls of a central portion 126 of the acoustic affinity filter 110. FIG. 1B illustrates operation of the system 100 in cycle in which the microbeads 120 are preloaded in the acoustic affinity filter 110 rather than being initially present in the bioreactor 112.

FIGS. 1C-1E show the system 100 being operated in cycle in which the microbeads 120 are present in the bioreactor 112 during incubation and captured with attached target compounds in the capture mode (see FIG. 1C). After elution (see FIG. 1D), the functionalized material is returned to the bioreactor (see FIG. 1E).

The acoustic transducer 122 includes a vibrating material such as a piezoelectric material. When operated, the acoustic transducer 122 can create a plane wave distribution, a multidimensional acoustic field distribution, or a combination of plane wave and multidimensional acoustic field distribution. The resulting acoustic wave distribution between the acoustic transducer 122 and the reflector 124 can give rise to a standing wave distribution with a spatial pattern of acoustic radiation force. In FIG. 1B, the acoustic wave distribution represented by the curved lines 117 in the central portion 126 of the acoustic affinity filter 110.

The acoustic transducer 122 can be driven by a voltage signal, e.g., a pulsed voltage signal with a frequency of 100 kHz to 10 MHz, such that the vibrating material is vibrated at a higher order vibration mode to generate an acoustic wave that is reflected by the reflector 124 to create a standing wave (from a plane wave, a multidimensional wave, or a combination of a plane wave and a multidimensional wave). The multidimensional acoustic wave may be generated by a higher order mode perturbation of the vibrating material. In some cases, the acoustic wave is a multiple component wave generated by the higher order mode perturbation of the vibration material. In some cases, the acoustic wave is a combination of a multiple component wave generated by the higher order mode perturbation of the vibration material and a planar wave generated by a piston motion of the vibration material. The higher order vibration mode can be in a general formula (m, n), where m and n are an integer and at least one of m or n is greater than 1. In this example, the acoustic transducer 122 vibrates in higher order vibration modes than (2, 2), which produce more nodes and antinodes, resulting in three-dimensional standing waves in the acoustic affinity filter 110.

The acoustic transducer 122 can be variably configured to generate higher order vibration modes. In some implementations, the vibrating material is configured to have an outer surface directly exposed to a fluid layer, e.g., the mixture of microcarriers and cultured cells in a fluid flowing through the flow chamber. In some implementations, the acoustic transducer includes a wear surface material covering an outer surface of the vibrating material, the wear surface material having a thickness of a half wavelength or less and/or being a urethane, epoxy, or silicone coating, polymer, or similar thin coating. In some implementations, the acoustic transducer includes a housing having a top end, a bottom end, and an interior volume. The vibrating material can be positioned at the bottom end of the housing and within the interior volume and has an interior surface facing to the top end of the housing. In some examples, the interior surface of the acoustic material is directly exposed to the top end housing. In some examples, the acoustic transducer includes a backing layer contacting the interior surface of the acoustic material, the backing layer being made of a substantially acoustically transparent material. One or more of the configurations can be also combined in the acoustic transducer 122 to be used for generation of a multi-dimensional acoustic standing wave.

The acoustic radiation force can have an axial force component and a lateral force component that are of the same order of magnitude. The spatial pattern may manifest as periodic variations of density. More specifically, pressure node planes and pressure anti-node planes can be created in a fluid medium that respectively correspond to peak acoustic radiation force planes and floor acoustic radiation force planes. In FIG. 1B, the peaks and floors of the acoustic radiation force planes correspond to locales where beads 120 are trapped. This spatial pattern of nodes and antinodes may function much like a filter in the fluid medium to trap particles of a particular size or size range, while particles of a different size or size range may not be trapped. In some configurations, the spatial pattern can be configured, for example, by adjusting the insonification frequency, power of the transducer, or fluid velocity, to allow some material to freely flow through while trapping some particular functionalized materials, such as microcarriers with specific antigen configurations. In other words, the acoustic standing wave may be tuned specifically to the microcarrier with the functionalized surface.

Some systems are implemented other functionalized materials or microcarriers (e.g., paramagnetic beads or hydrogel particles). The microcarriers can be designed with a surface chemistry which allows for attachment and growth of anchorage dependent cell lines. The microcarriers can be made from a number of different materials, including DEAE (N,N-diethylaminoethyl)-dextran, glass, polystyrene plastic, acrylamide, collagen, and alginate. The microcarrier materials, along with different surface chemistries, can influence cellular behavior, including morphology and proliferation. Surface chemistries for the microcarriers can include extracellular matrix proteins, recombinant proteins, peptides, and positively or negatively charged molecules. Microcarriers describes materials with a characteristic dimension (e.g., average diameter, length of primary axis, length, or width) of between 01. and 1000 microns.

In some implementations, the microcarriers are formed by substituting a cross-linked dextran matrix with positively charged DEAE groups distributed throughout the matrix. This type of microcarrier can be used for established cell lines and for production of viruses or cell products from cultures of primary cells and normal diploid cell strains.

In some implementations, the microcarriers are formed by chemically coupling a thin layer of denatured collagen to the cross-linked dextran matrix. Since the collagen surface layer can be digested by a variety of proteolytic enzymes, it provides opportunities for harvesting cells from the microcarriers while maintaining maximum cell viability and membrane integrity.

In some configurations, a functionalized surface of the microcarrier may include a specific antibody ligand. This specific antibody ligand may have affinity for a specific antigen (such as CD34 or CK8) that permits to bind a specific type of cell (a stem cell or a CTC for these antigens, respectively). The trapped microcarriers with the affinity modified surface are utilized as an acoustic fluidized bed filter where specific proteins, antibodies or cells are attracted to the surface of the functionalized microcarrier and held along with the microcarrier in the acoustic standing wave.

Examples of the affinity centers include enzymes, antibodies, aptamers, oligonucleotides, streptavidin, etc. Oligonucleotide may be synthesized using either “classic” RNA or DNA monomers, or nucleic acid mimics (e.g. PNA, LNA, etc.), or the mixture of both. The objects of interest that are specific to the affinity centers attached to the microcarriers become bound to the affinity centers of the microcarriers that are trapped in the acoustic standing wave. The objects of interest can include biomolecules, viruses, and live cells. To bind to the affinity centers, they may carry a complementary determinant, such as biotin for streptavidin, antigen to antibody, complimentary oligonucleotide, etc. By this method, biomolecules, viruses, or live cells of interest in a cellular and particulate fluid system, such as blood, may be selectively removed from the secondary fluid system. The cells of interest include, for example, Chinese Hamster Ovary (CHO) cells and plasma cells. Examples of materials of interest include, for example, immunoglobulins, monoclonal antibodies and recombinant proteins, biological objects conjugated with complementary determinants, such as labeled proteins, viruses and biomolecules with complementary epitopes, etc.

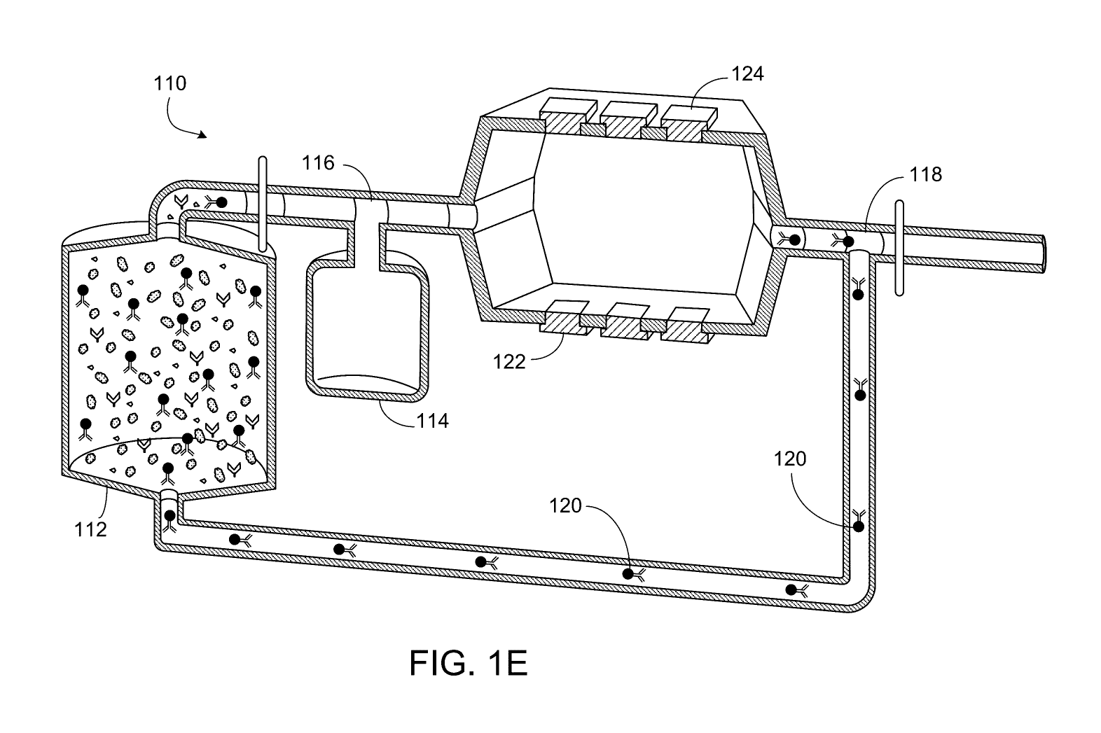

FIG. 2 illustrates a process 200 for extracting target compounds (e.g., the materials or monoclonal antibodies 113) from a carrier fluid using functionalized material (e.g., the microbeads 120). The functionalized material is retained in a liquid-filled chamber (e.g., acoustic affinity filter) at peak and valley locales within an acoustic standing wave field (step 210). Fluid containing target compounds flows into the liquid-filled chamber where functionalized material has been retained by acoustic insonification such that the target compounds are filtered from the fluid by the retained functionalized material (step 212). The fluid is processed inside the chamber to cause the trapped portions of target compounds to elute (step 214) and the eluted target compounds are collected (step 216).

For example, the process 200 can be used to capture target compounds using the system 100 shown in FIGS. 1A-1E. Before operation of the system 100, the acoustic affinity filter 110 is preloaded with the microbeads 120 of chromatography resin that has affinity to the materials being produced. The microbeads 120 are retained in the liquid-filled acoustic affinity filter 110 at peak and valley locales within an acoustic standing wave field 117 indicated by the wavy lines in FIG. 1B-1D.

The three-way valve 116 and the three-way valve 118 are closed while the bioreactor is operated to cause the cells 111 contained in the bioreactor 112 to produce materials 113. The switch over to filtering/capturing will happen on a continuous basis for perfusion and for fed batch bioreactors, when the desired production of proteins, viability of cells and ancillary cell debris reach specified conditions. In today's bioreactor processes, higher concentrations of cells and longer fermentation times result in higher drug titers and greater product yields. These bioreactor conditions reduce cell viability, increase cell debris, and raise concentrations of organic constituents in the cell broths. The amorphous, colloidal nature of these components tends to complicate the separation process. The choice of a clarification technology will also take into account any requirements for integration with downstream processes such as chromatography and ultrafiltration. A filtration step such as depth filtration may be utilized to relieve the load on downstream filters and processes.

After a desired level of materials 113 has been reached, the three-way valve 116 is operated to provide a fluid connection between the outlet of the bioreactor 112 and the inlet of the acoustic affinity filter 110. For example, the system 100 is switched (automatically or manually) to capture mode when target compounds reach a concentration of 5 grams/L concentration. Some systems are configured to switch to capture mode when target compounds reach a concentration of between 0.5 and 20 grams/L (e.g., more than 1 grams/L, more than 2.5 grams/L, more than 5 grams/L, more than 7.5 grams/L, more than 10 grams/L, more than 15 grams/L, less than 17.5 grams/L, less than 15 grams/L, less than 10 grams/L, less than 5 grams/L, or less than 2.5 grams/L).

The three-way valve 118 is operated to provide a fluid connection between the outlet of the acoustic affinity filter 110 and the inlet of the bioreactor 112. The culture suspension fluid is circulated through the resulting fluid circuit by an inline pump (not shown). Some systems use other pumps or fluid transfer mechanisms to cause the fluid to flow.

As the culture suspension fluid passes through the acoustic affinity filter 110, the cells 111 continue around the fluid circuit with the culture suspension fluid and are returned to the bioreactor. The acoustic affinity filter 110 is tuned to provide nodes with a characteristic dimension (e.g., width, length, or diameter) of 100-500 microns (e.g., between 200 and 400 microns, greater than 200 microns, greater than 250 microns, greater than 300 microns, greater than 350 microns, greater than 200 microns, greater than 200 microns, greater than 200 microns, less than 500 microns, less than 450 microns, less than 400 microns, less than 350 microns, less than 300 microns) and spacing between nodes (e.g., from the edge of one node to the edge of an adjacent node) of 25-150 microns (e.g., between 50 and 100 microns, greater than 25 microns, greater than 50 microns, greater than 75 microns, greater than 100 microns, less than 150 microns, less than 125 microns, less than 100 microns, less than 75). Acoustic affinity filters with these properties can facilitate easy passage of the cells 111 and other non-target materials.

For example, the acoustic affinity filter 110 is tuned and preloaded to maintain microbeads 120 at a volume ratio of the volume occupied by microbeads 120 divided by total volume of the portion of filter region 126 containing microbeads 120 of less than 50% (e.g., less than 40%, less than 30%, less than 20%, less than 15%, less than 10%). This volume ratio reflects low density arrangement of the microbeads and facilitates easy passage of the cells 111, cell debris, and nonspecific proteins and is lower than the volume ratio in a typical packed column. The lower volume ration and increased spacing between decreases the likelihood that non-target biomaterials will clog flow paths between the functionalized particles. Recirculating media containing the target biomaterials in effect increases the capture surface area of the apparatus by passing free target biomaterials past the functionalized particles multiple times. The reduced contact of non-target biomaterials can help preserve non-target biomaterials such as cells being used to produce, for example, proteins. The technology described here can be used in high density cell culture, new research applications, large production culture volumes, e.g., more than 1,000 liters, efficient monitoring and culture control, reduction of costs and contamination in cell culture applications.

The materials 113 are much smaller than the cells 111. Some of the materials 113 come in contact with and are retained by the microbeads 120. However, some of the materials 113 continue around the fluid circuit with the culture suspension fluid and are returned to the bioreactor 112. The system 100 compensates for this effect of the reduced surface area per volume of the microbeads 120 relative to a packed column by passing the suspension fluid and contained materials 113 through the acoustic affinity filter multiple times (e.g., 4, 6, 8, 10, or more times). During this capture process, the bioreactor 112 is operated to continue to produce more materials 113. In some systems, the functionalized material is suspended in the reactor, incubated in the culture to collect the target compounds before the culture suspension is pumped through the acoustic affinity filter which collects the functionalized material and the associated target compounds.

The 3-way valve 116 is operated to close the outlet piping from the bioreactor 112 and open a fluid connection between the elution buffer reservoir 114 and the acoustic affinity filter 110 to switch the system from capture mode to elution mode. The three-way valve 118 is operated to close the inlet piping to the bioreactor 112 and to open a fluid connection between the acoustic affinity filter 110 and a collection outlet of the system 100. The elution buffer releases the materials 113 from the microbeads 120 and carries the materials 113 out of the system 100 through the collection outlet of the system 100. The microbeads 120 can be restored and held in the acoustic affinity filter for the next operation cycle of the system 100. In systems in which the functionalized material is suspended in the reactor, the microbeads 120 can be released and returned back into the bioreactor 112 (see, e.g., FIGS. 1C-1E).

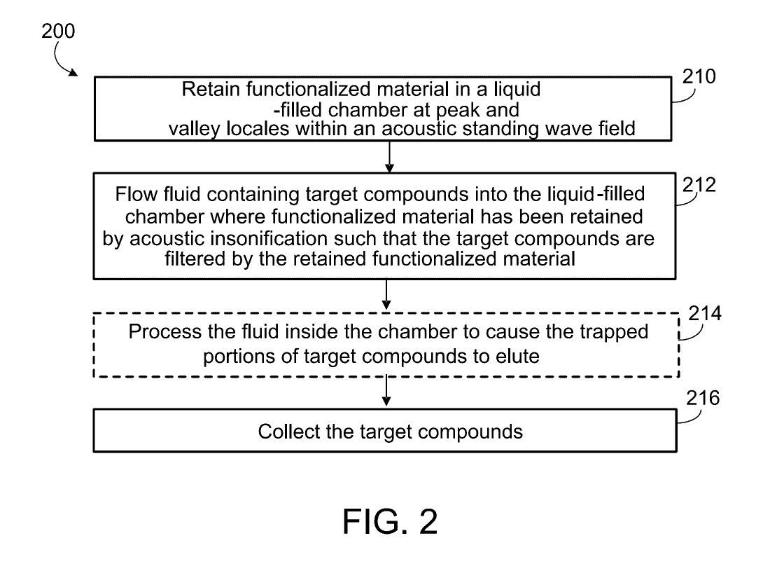

FIG. 3 is a plan view of a portion of an experimental setup built to demonstrate the capture and suspension of chromatography beads in an affinity acoustic affinity filter. A 1″×1″×1″ system 300 was built with two transducers 310 adjacent to each other and complementary steel reflectors 312 across from them. This system also had a steel bottom side and the top side was left open to the air. The system was filled to its holding capacity with clean, deionized water. The exemplary 1 inch by 1 inch acoustic affinity filter driven at 2.3 MHz and tuned to provide nodes ˜337 microns wide with ˜77 micron spacing was observed to effectively maintain polystyrene microbeads for use in affinity capture of passing target compounds.

Sepharose chromatography microbeads conjugated with Protein A with diameter 34 micrometers were extracted from HiTrap Protein A HP 1 mL columns from GE Life Sciences. Protein A binds to monoclonal and polyclonal antibodies. Therefore, if these microbeads were placed in a solution containing such antibodies, they will bind tightly to the antibodies, separating them from the solution. These microbeads 320 were added to the water in the system.

The microcarriers or microbeads may have a positive or negative acoustic contrast factor. For example, microcarriers with a reflective core that bounces incident acoustic standing waves have a positive contrast factor. Such microcarriers may be driven by the acoustic radiation force to the pressure nodal hot spots within the pressure planes. Microcarriers with an absorbent core may accept incident acoustic standing waves more than bouncing these waves. Such microcarriers may have a negative contrast factor, and may be driven by the acoustic radiation force to the pressure anti-nodal planes. The cells, on the other hand, are not trapped by the insonification process and can flow with the fluid medium.

The transducer was then powered at a constant voltage of 45V at 2.23 MHz fixed frequency. As predicted, the microbeads 320 aligned themselves along trapping lines that closely mirror expected patterns predicted using finite element analysis.

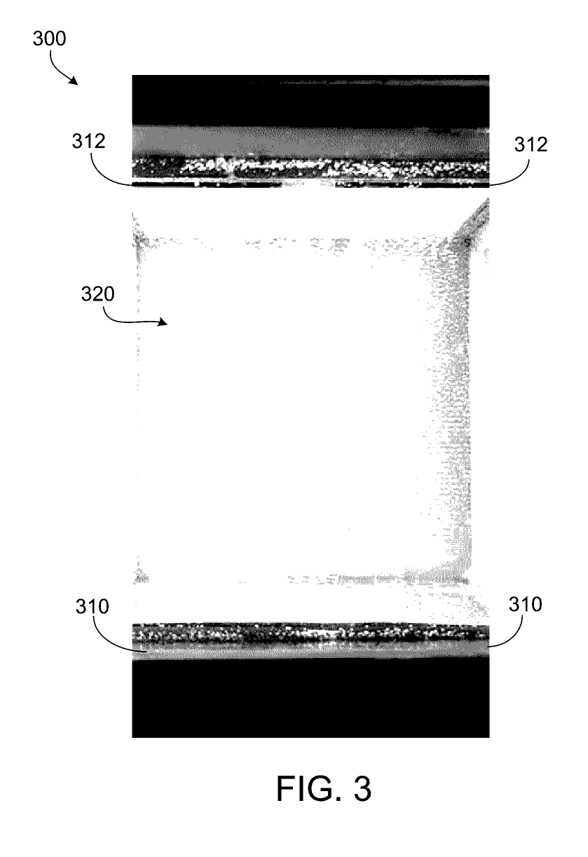

FIG. 4 illustrates a process 500 in which the functionalized material is incubated directly in a cell culture suspension within the bioreactor 112. The microbeads 120 (or other functionalized material) bind the target proteins during the incubation within the bioreactor 112. The cell culture suspension from the bioreactor 112 is pumped through the acoustic affinity filter 110. The microbeads 120 and attached target proteins are retained in the acoustic affinity filter while cells and other material go through the acoustic affinity filter 110.

Depending on the user's goals, the cells may be either discarded or returned into the bioreactor (510). As to the beads, there are multiple options. For example, in one approach, the transducer of the acoustic affinity filter 110 is turned off releasing a slurry containing the microbeads 120 and attached target proteins (512). The slurry is recovered and further processed outside of the acoustic affinity filter. In another approach, the acoustic affinity filter 110 with the microbeads 120 inside can be used similarly to a chromatography column in a dedicated cycle (514).

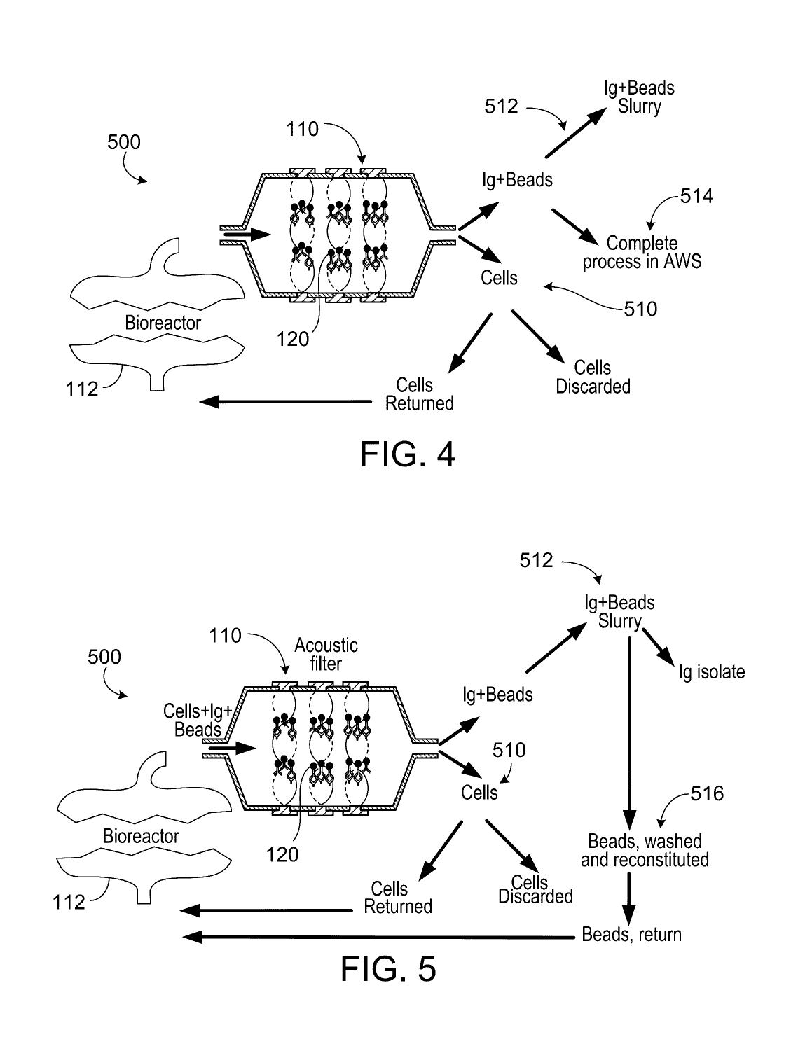

FIG. 5 illustrates an embodiment of process 500 in which the slurry is loaded in a chromatography column and processed in the way similar to a regular chromatographic procedure. It typically includes packing the slurry, washing the beads, eluting the protein, and reconstituting the beads. Washing is typically performed with a buffered solvent that removes nonspecifically bound matters, while the protein remains specifically bound to the beads. Elution removes the protein from the beads. Depending on the affinity or binding centers, elution can be performed by change of pH and/or of ionic strength, by inactivation of the affinity center (e.g. denaturation of the complex-forming protein), by excess of a competing ligand, etc. This process essentially inactivates the affinity centers. Alternatively, the recovered slurry can be placed on top of a filter and washed with similar solvents as in the chromatography column approach.

After protein recovery, the beads can be discarded or returned into the reactor. To reuse them, the beads must be reconstituted (the affinity centers must be reactivated) (516. To reconstitute them, the beads are washed with an appropriate solvent (e.g. a buffer with low ionic strength for ion-exchange beads).

The beads can be recovered from the acoustic affinity filter 110 either in batch or continuous mode. In a batch mode, the flow of the cell suspension is interrupted and the protein-loaded beads are either collected through the bottom port or washed out through the permeate port. In a continuous mode, the acoustic trapping regime is adjusted so that the retained beads do not escape the acoustic affinity filter with the permeate flow, but instead are concentrated, precipitate, and are collected through the bottom (a concentrate port).

The slurry can be collected either sequentially or in a staggered mode. In the former, the cell suspension flow is interrupted for the time of the slurry recovery. Therefore, this process can be performed with a single unit. In the latter, the cell suspension flow is redirected to another unit, while the first one is in the slurry recovery mode.

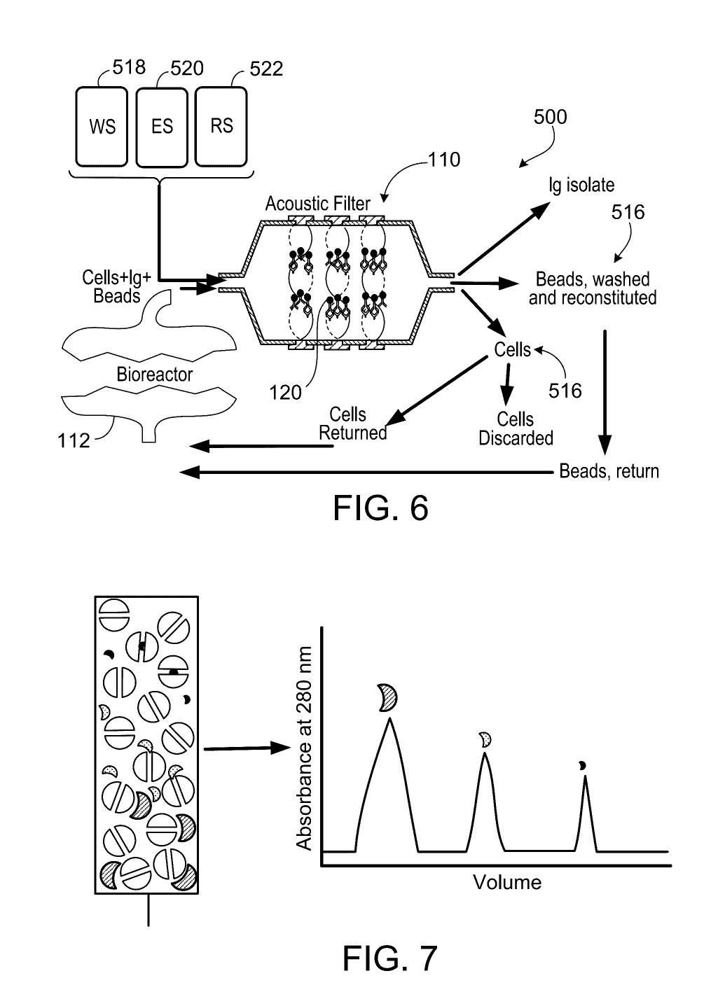

FIG. 6 illustrates an embodiment of process 500 in which the acoustic affinity filter 110 with the microbeads inside can be used similarly to a chromatography column in a dedicated cycle (514). In this embodiment, the beads are processed in situ, without removal from the acoustic affinity filter 110. The retained beads are treated with washing, elution, and reconstitution solvents (518, 520, and 522, respectively) in the same manner as described above. During this operation, the cell suspension flow is either interrupted or redirected to another acoustic unit to continue the bead recovery process.

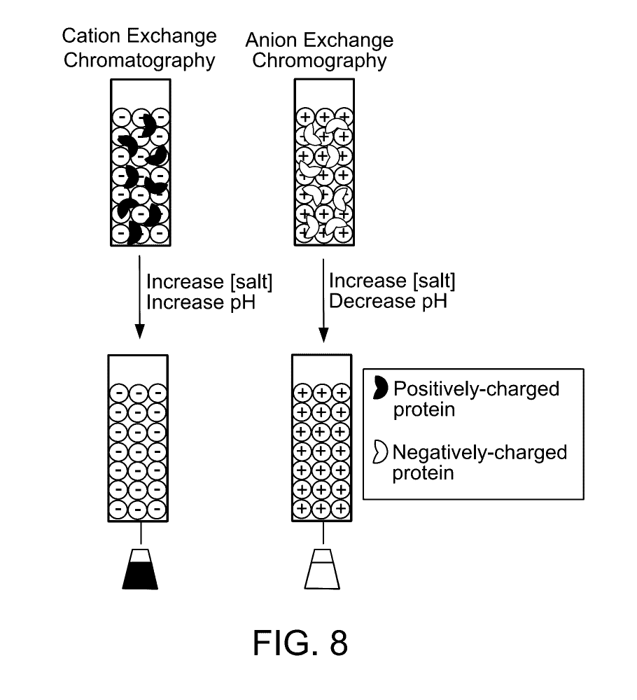

FIG. 7 illustrates using size exclusion chromatography for post processing the slurry of functionalized material and attached target compounds in which different target compounds have different sizes. This approach can be used for separating trapped biomolecules, viruses, or live cells of interest from functionalized material with several regions of interest for affinity separation. For example, the functionalized material can include portions that bind different target compounds or non-selectively bind multiple compounds. As a solvent releases bound compounds from the functionalized material, larger proteins elute first, as they are unable to enter the pores of the adsorbent/analyte complex and have a more direct path through the column. Smaller proteins can enter the pores, have a more convoluted path and, thus, take longer to traverse the matrix and elute from the column.

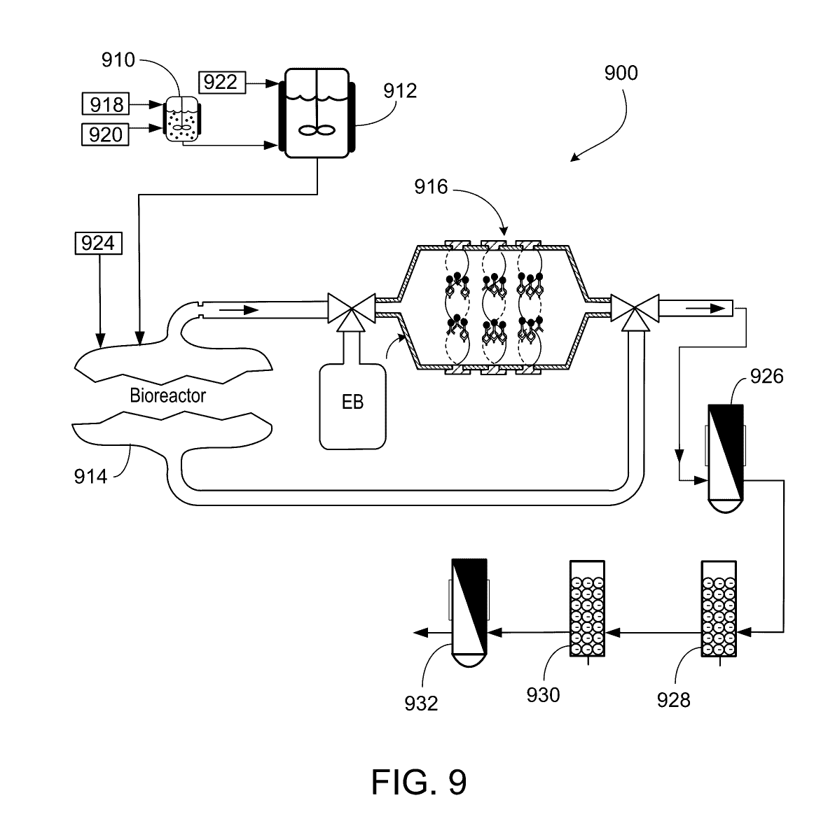

FIG. 8 illustrates using ion exchange for post processing the slurry of functionalized material and attached target compounds. In this approach, the target compounds are released from the functionalized material, e.g., by increasing the ionic strength of the buffer or by adjusting the pH of the buffer. At high ionic strength, proteins are partially desolvated, causing them to adopt alternate conformations in which normally buried hydrophobic residues are more exposed. These residues can then form hydrophobic interactions with the hydrophobic functional groups conjugated to a matrix. Lowering the ionic strength causes the protein to refold into its native conformation, burying its hydrophobic residues. This decreases hydrophobic interactions between the protein and stationary phase, facilitating protein elution.

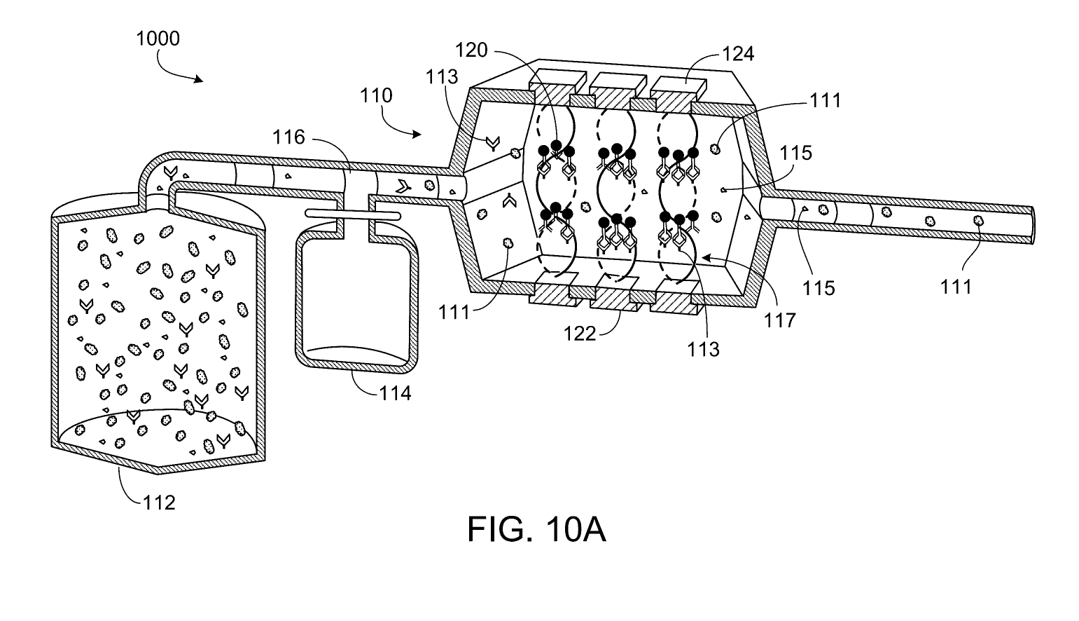

FIG. 9 illustrates a system 900 for producing therapeutic proteins that incorporates a bioreactor—acoustic affinity filter circulation loop like the system 100 show in FIG. 1. The system 900 includes a first seed bioreactor 910, a second seed bioreactor 912, and a production bioreactor 914 that utilizes a population of cells expressing therapeutic proteins such as monoclonal antibodies and recombinant proteins. An acoustic affinity filter 916 captures the monoclonal antibodies and recombinant proteins and several filters and columns are used for post-processing.

The first seed bioreactor 910 (a.k.a., the N-2 bioreactor) is a 300 liter bioreactor that receives input from bag reactors 918 used for initial cell production and from a media preparation system 920. The second seed bioreactor 912 (a.k.a., the N-1 bioreactor) is a 2,000 liter bioreactor that receives input from the first seed bioreactor 910 and a media preparation system 922. The production bioreactor 914 (a.k.a., the N bioreactor) is a 15,000 liter that receives input from the second seed bioreactor 912 and a media preparation system 924. Other systems can include different numbers of bioreactors and/or bioreactors with different sizes than those included in the system 900.

The production bioreactor 914 and the acoustic affinity filter 916 are included in a flow loop that also includes the other components shown in FIG. 1 and described in the associated text. The loop is operated as described above to produce and capture the target compounds on activated material inside the acoustic affinity filter 916. The acoustic affinity filter 916 provides the cellular clarification and harvest from the bioreactor, and yields a relatively pure product that, while mostly pure, still requires removal of a small proportion of process and product related impurities.

The system 900 includes a polishing filter 926 configured to remove any remaining particles that are larger than 0.2 microns, an ion exchange chromatography column 928, a hydrophobic interaction column 930, and a final polishing filter 932. Some systems include different post capture processing components.

The ion exchange chromatography column 928 removes non-target proteins using incorporating cation and anion exchange chromatography. As discussed above with reference to FIG. 7, specific proteins (either target or non-target proteins) are attached to the column media.

The hydrophobic interaction column 930 uses the properties of hydrophobicity to separate proteins from one another. In this column, hydrophobic groups such as phenyl, octyl, or butyl, are attached to the stationary column. Proteins that pass through the column that have hydrophobic amino acid side chains on their surfaces are able to interact with and bind to the hydrophobic groups on the column. In this process of chromatography, separations are often designed using the opposite conditions of those used in ion exchange chromatography. In this separation, a buffer with a high ionic strength, usually ammonium sulfate, is initially applied to the column. The salt in the buffer reduces the solvation of sample solutes thus as solvation decreases, hydrophobic regions that become exposed are adsorbed by the medium), mixed mode chromatography or hydroxyapatite chromatography—HAP. The mechanism of HAP is complicated and involves nonspecific interactions between negatively charged protein carboxyl groups and positively charged calcium ions on the resin, and positively charged protein amino groups and negatively charged phosphate ions on the resin. Basic or acidic proteins can be adsorbed selectively onto the column by adjusting the buffer's pH; elution can be achieved by varying the buffer's salt concentration also may be chosen. These steps provide additional separation of viral, host cell protein and DNA materials, as well as removing aggregates, unwanted product variant species and other minor contaminants.

The final polishing filter 932 provides diafiltration using ultrafiltration membranes to completely remove, replace, or lower the concentration of salts or solvents from solutions containing proteins, peptides, nucleic acids, and other biomolecules. The process selectively utilizes permeable (porous) membrane filters to separate the components of solutions and suspensions based on their molecular size into a final formulation buffer.

Additionally, some systems include a low pH hold post Protein A chromatography and a viral filtration step to achieve sufficient viral clearance.

A number of embodiments of the invention have been described. Nevertheless, it will be understood that various modifications may be made without departing from the spirit and scope of the invention.

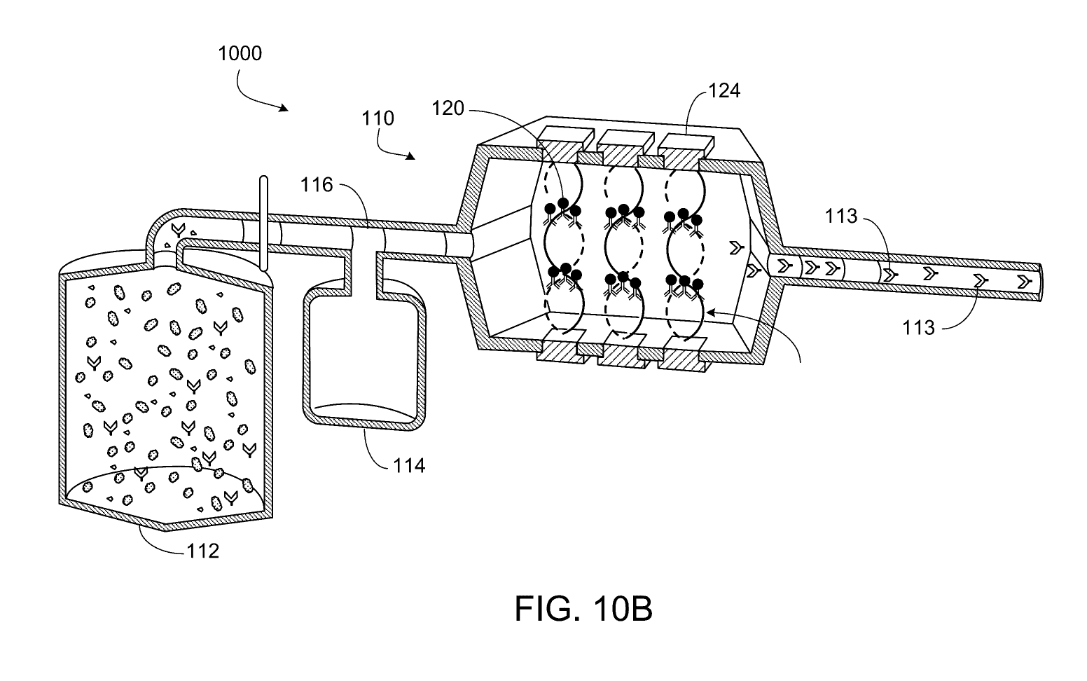

For example, FIGS. 10A and 10B show the operation of a system 600 for producing target compounds such as, for example, monoclonal antibodies and recombinant proteins. The system 1000 is similar to the system 100 shown in FIG. 1A but does not include a recirculation loop from the outlet of the acoustic affinity filter 110 back to the bioreactor 112. FIG. 10A shows the system 1000 in capture mode with target compounds 113, plasma cells 111, and debris flowing from the bioreactor 112 to the acoustic affinity filter 110. In the acoustic affinity filter 110, the functionalized particles (e.g., microbeads 120) capture the target compounds 113 while the plasma cells 111 and debris 115 flow through. FIG. 10B shows the system 1000 in elution mode with the 3-way valve 116 is operated to close the outlet piping from the bioreactor 112 and to open a fluid connection between the elution buffer reservoir 114 and the acoustic affinity filter 110. The target compounds are released from the functionalized material and collected at the outlet of the system 1000.

Gork'ov's model is for a single particle in a standing wave and is limited to particle sizes that are small with respect to the wavelength of the sound fields in the fluid and the particle. It also does not take into account the effect of viscosity of the fluid and the particle on the radiation force. As a result, this model cannot be used for the macro-scale ultrasonic separators discussed herein since particle clusters can grow quite large. A more complex and complete model for acoustic radiation forces that is not limited by particle size was therefore used. The models that were implemented are based on the theoretical work of Yurii Ilinskii and Evgenia Zabolotskaya as described in AIP Conference Proceedings, Vol. 1474-1, pp. 255-258 (2012). These models also include the effect of fluid and particle viscosity, and therefore are a more accurate calculation of the acoustic radiation force.

When acoustic standing waves propagate in liquids, the fast oscillations may generate a non-oscillating force on particles suspended in the liquid or on an interface between liquids. This force is known as the acoustic radiation force. The force originates from the non-linearity of the propagating wave. As a result of the non-linearity, the wave is distorted as it propagates and the time-averages are nonzero. By serial expansion (according to perturbation theory), the first non-zero term will be the second-order term, which accounts for the acoustic radiation force. The acoustic radiation force on a particle, or a cell, in a fluid suspension is a function of the difference in radiation pressure on either side of the particle or cell. The physical description of the radiation force is a superposition of the incident wave and a scattered wave, in addition to the effect of the non-rigid particle oscillating with a different speed compared to the surrounding medium thereby radiating a wave. The following equation presents an analytical expression for the acoustic radiation force on a particle, or cell, in a fluid suspension in a planar standing wave.

$${F}_{R} = \frac{3 π {P}_{0}^{2} {V}_{P} {β}_{m}}{2 λ} ϕ ( β , ρ ) sin ( 2 kx ) \qquad ( 1 )$$

where βₘ is the compressibility of the fluid medium, ρ is density, φ is acoustic contrast factor, Vₚ is particle volume, λ is wavelength, k is 2π/λ, P₀ is acoustic pressure amplitude, x is the axial distance along the standing wave (i.e., perpendicular to the wave front), and

$$ϕ ( β , ρ ) = \frac{5 {ρ}_{ρ} - 2 {ρ}_{m}}{2 {ρ}_{ρ} + {ρ}_{m}} - \frac{{β}_{ρ}}{{β}_{m}}$$

where ρₚ is the particle density, ρₘ is the fluid medium density, βₚ is the compressibility of the particle, and βₘ is the compressibility of the fluid medium.

For a multi-dimensional standing wave, the acoustic radiation force is a three-dimensional force field, and one method to calculate the force is Gor'kov's method, where the primary acoustic radiation force F_{R} is defined as a function of a field potential U, F_{V}=−∇(U), where the field potential U is defined as

$$U = {V}_{0} [ \frac{〈 {p}^{2} ( x , y , t ) 〉}{2 {ρ}_{f} {c}_{f}^{2}} {f}_{1} - \frac{3 {ρ}_{f} 〈 {v}^{2} ( x , y , t ) 〉}{4} {f}_{2} ]$$

and f₁ and f₂ are the monopole and dipole contributions defined by

$${f}_{1} = 1 - \frac{1}{{Λσ}^{2}} {f}_{2} = \frac{2 ( Λ - 1 )}{2 Λ + 1} , where$$

$$σ = \frac{{c}_{ρ}}{{c}_{f}} Λ = \frac{{ρ}_{ρ}}{{ρ}_{f}} {β}_{f} = \frac{1}{{ρ}_{f} {c}_{f}^{2}}$$

where p is the acoustic pressure, u is the fluid particle velocity, Λ is the ratio of cell density ρₚ to fluid density ρ_{f}, σ is the ratio of cell sound speed cₚ to fluid sound speed c_{f}, Vₒ is the volume of the cell, and < > indicates time averaging over the period of the wave.

Gork'ov's model is for a single particle in a standing wave and is limited to particle sizes that are small with respect to the wavelength of the sound fields in the fluid and the particle. It also does not take into account the effect of viscosity of the fluid and the particle on the radiation force. As a result, this model cannot be used for the macro-scale ultrasonic separators discussed herein since particle clusters can grow quite large. A more complex and complete model for acoustic radiation forces that is not limited by particle size was therefore used. The models that were implemented are based on the theoretical work of Yurii Ilinskii and Evgenia Zabolotskaya as described in AIP Conference Proceedings, Vol. 1474-1, pp. 255-258 (2012). These models also include the effect of fluid and particle viscosity, and therefore are a more accurate calculation of the acoustic radiation force.

Accordingly, other embodiments are within the scope of the following claims.

1. A method of separating a first biomaterial from a second biomaterial, the method comprising:
retaining functionalized material in a liquid-filled chamber at locales within an acoustic standing wave field, the locales distributed inside the chamber where acoustic pressure amplitude is either elevated compared to when the acoustic transducer is turned off, or substantially identical to when the acoustic transducer is turned off;
flowing a culture suspension containing the first biomaterial and the second biomaterial into the liquid-filled chamber where functionalized material has been retained by acoustic insonification such that at least portions of the first biomaterial with features complementary to the functionalized material become bound to the functionalized material while other portions of the culture suspension containing the second material pass through the chamber, the first material being at least two orders of magnitude smaller than the second material; and
subsequently releasing the portion of the first biomaterial bound to the functionalized material from the liquid filled chamber.

2. The method of claim 1, wherein flowing the culture suspension containing the first and the second biomaterials into the liquid-filled chamber comprises circulating the cell culture containing the first and second biomaterials such that the first and second biomaterials flow through the locales distributed inside the chamber where acoustic pressure amplitude is either elevated compared to when the acoustic transducer is turned off, or substantially identical to when the acoustic transducer is turned off more than once.

3. The method of claim 1, wherein the first biomaterial comprises biomolecules.

4. The method of claim 3, wherein the first biomaterial biomolecules include monoclonal antibodies, recombinant proteins, or both.

5. The method of claim 1, wherein the second biomaterial comprises cells.

6. The method of claim 5, wherein the cells comprise Chinese Hamster Ovary (CHO) cells.

7. The method of claim 1, wherein subsequently releasing the portion of the first biomaterial bound to the functionalized material from the liquid filled chamber comprises releasing the portion of the first biomaterial bound to the functionalized material from the liquid filled chamber and releasing the functionalized material from the liquid filled chamber.

8. The method of claim 1, wherein subsequently releasing the portion of the first biomaterial bound to the functionalized material from the liquid filled chamber comprises processing the culture suspension inside the chamber to cause the first biomaterial that are bound to the functionalized material to elute from the liquid filled chamber while the functionalized materials are maintained in the liquid filled chamber.

9. The method of claim 1, wherein the portion of the first biomaterial form antigen-antibody interactions with binding sites on the functionalized material.

10. The method of claim 1, wherein the portion of the samples become bound to the functionalized material when a ligand of the portions of the samples is conjugated to a matrix on the functional material.

11. The method of claim 1, wherein the functionalized material comprises one of: functionalized microbeads, functionalized paramagnetic beads, functionalized hydrogel particles.

12. The method of claim 11, wherein the functionalized material include a particular antigen ligand that has affinity for a corresponding antibody specific to a particular protein molecule.

13. The method of claim 11, wherein the functionalized material comprises microbeads with a positive or negative acoustic contrast factor.

14. The method of claim 1, further comprising: passing the culture suspension through a size exclusion column wherein the bound portions of the first biomaterial of a first hydrodynamic radius elutes before the bound portions of the first biomaterial with a second hydrodynamic radius when the first hydrodynamic radius is larger than the second hydrodynamic radius.

15. The method of claim 1, further comprising: increasing an ionic strength of the culture suspension to cause the portion of the first biomaterial that are bound to the functionalized material to elute or adjusting a pH level of the culture suspension to cause the portion of the first biomaterial that are bound to the functionalized material to elute.

16. The method of claim 1, further comprising: lowering an ionic strength of the culture suspension to cause the portion of the first biomaterial that are bound to the functionalized material to refold into a native formation such that a hydrophobic interaction between the portion of the first biomaterial and the functionalized material is decreased.

17. The method of claim 14, further comprising: determining a quantitative level of the portion of the first biomaterial eluted to form a chromatography readout.

18. The method of claim 17, wherein determining the quantitative level comprises determining a mass or a volume.

19. The method of claim 17, wherein determining the quantitative level comprises measuring an optical absorption index of the portion of eluted first biomaterial.

20. A system for separating a first biomaterial from a second biomaterial, the system comprising:
functionalized material with features complementary to the first biomaterial;
a flow chamber having a first wall and a second wall opposite to each other, and configured to receive fluid containing the functionalized material; and
an acoustic transducer mounted on the first wall and a reflector mounted on the second wall such that when the acoustic transducer is turned on, a multi-dimensional acoustic field is created inside the chamber that includes first spatial locales where acoustic pressure amplitude is elevated from when the acoustic transducer is turned off, and second spatial locales where acoustic pressure amplitude is substantially identical to when the acoustic transducer is turned off, the acoustic transducer tuned to trap the functionalized material at the first or second locales of the multidimensional acoustic field;
wherein a volume occupied by the functionalized material divided by total volume of a region containing the functionalized material is less than 50%.

21. The system of claim 20, wherein a characteristic size of the first biomaterial is at least two orders of magnitude smaller than a characteristic size of the second biomaterial.

22. The apparatus of claim 21, further comprising: an analysis bin configured to receive the portion of the first biomaterial bound to the functionalized material and subsequently eluted such that a chromatography measurement of the portion of the first biomaterial is obtained.

23. The system of claim 22, further comprising: a size exclusion column coupled to the flow chamber.

24. The system of claim 22, further comprising: a hydrophobic interaction chromatography column coupled to the flow chamber.

25. The system of claim 22, further comprising: an ion exchange chromatography column coupled to the flow chamber.

26. The system of claim 22, further comprising: a mass spectrometer to measure an amount of the portion of the first biomaterial in the analysis bin.

27. The system of claim 22, further comprising: an optical spectrometer to measure an amount of the portion of the first biomaterial in the analysis bin.
